(19) United States
(12) Patent Application Publication
(10) Pub. No.: US 2013/0302888 A1
(43) Pub. Date: Nov. 14, 2013

(54) CELL CULTURE METHOD AND SYSTEM FOR ESTABLISHING AN IN VITRO MODEL OF THE INTESTINAL BARRIER

(71) Applicants: Frederic Demarne, Marseille (FR); Vincent Jannin, Lyon (FR); Alf Lamprecht, Bonn (DE); Yann Pellequer, Besancon (FR); Arnaud Beduneau, Epenouse (FR)

(72) Inventors: Frederic Demarne, Marseille (FR); Vincent Jannin, Lyon (FR); Alf Lamprecht, Bonn (DE); Yann Pellequer, Besancon (FR); Arnaud Beduneau, Epenouse (FR)

(73) Assignee: GATTEFOSSE SAS, Saint-Priest (FR)

(21) Appl. No.: 13/978,764

(22) PCT Filed: Feb. 17, 2012
(86) PCT No.: PCT/EP12/52780

§ 371 Date: Jul. 9, 2013

Related U.S. Application Data
(60) Provisional application No. 61/443,778, filed on Feb. 17, 2011.

(30) Foreign Application Priority Data
Feb. 17, 2011 (FR) 1151296

(51) Int. Cl. C12N 5/071

(52) U.S. Cl. CPC C12N 5/0679
USPC 435/366; 435/373

(57) ABSTRACT

Cell culture process includes seeding a suitable culture medium with enterocytes and then, after a delay, seeding the medium containing the enterocytes that have begun to proliferate, with goblet cells.

### TECHNICAL FIELD

This invention focuses on the development of a coculture model, based on Caco-2 absorption cells and HT29 goblet cells, for testing the transport of active substances and nutrients in the intestine. The purpose of the proposed coculture model is to create cell culture models that are as realistic as possible and which can be adapted to the specific properties of different parts of the human intestinal epithelium.

### PRIOR STATE OF THE ART

The Caco-2 cell line was isolated from a human colorectal cancer. Caco-2 cells differentiate spontaneously into well-developed polarised monolayers of columnar absorption cells expressing a brush border with typical enzymes (e.g. alkaline phosphatase, sucrase-isomaltase, aminopeptidase) on their apical surface. Active transporters for amino acids, nucleosides, bile acid, vitamins, oligopeptides and monocarboxylic acids are expressed on the apical side. The cells in these monolayers are joined by tight intercellular junctions that restrict paracellular passage of drug molecules and ions.^{1,2} However, tight bonding of the enterocytes in these monolayers is more like that of the colon than of the small intestine, which results in paracellular permeability in these monolayers which is too low for hydrophilic molecules.¹³ In addition, P-glycoprotein or P-gp (encoded by the multidrug resistance gene-1) is strongly expressed in Caco-2 cells.

The HT29 cell line is derived from a human colon adenocarcinoma. Post-confluent cultures of HT29 cells form a heterogeneous multilayer in which the majority of cells are undifferentiated. A subpopulation of mucus-producing goblet cells (HT29-MTX) has been obtained from the parental HT29 cell line by gradual adaptation to methotrexate (MTX). Several passages of HT29 cells in exponential growth were incubated with increasing amounts of MTX (10⁻⁷, 10⁻⁶, 10⁻⁵ mol). This results initially in high mortality, but leads to subpopulations with stable growth rates. These cells do not need to be maintained in a medium containing MTX to differentiate into mucus-producing goblet cells after confluence.^{3,4,5} The thickness of the mucus layer which spreads over the whole apical surface is on average 50 to 150 μm. HT29-MTX cells at passage 13 are available for example from Thecla Lesuffleur of INSERM U 938.

Cultures of Caco-2 cells have been extensively studied to predict the absorption of drugs from the gastrointestinal tract, despite less than optimal conditions. Indeed, the P-gp activity in Caco-2 monolayers in standard culture conditions is much higher than in the human colon in vivo.^{6,7} In addition, TEER (Trans Epithelial Electrical Resistance) values are very high, greater than 250 ohm.cm², in comparison with the human intestine (12 to 69 ohm.cm²),⁷ which results in paracellular permeability of hydrophilic active substances that is too low. Furthermore, the role of the mucus layer naturally occurring on the luminal side of the intestinal barrier is not taken into account in evaluating the permeability of active substances.^{8,9,10}

Proposals have been made to modify Caco-2 cells in an attempt to get closer to the in vivo situation. The document EP 1 158 045 describes a strain derived from Caco-2 with increased expression of cytochrome CYP3A4.

Furthermore, it has been proposed that cocultures could be made combining absorption cells or enterocytes and goblet cells. The first studies were by Wikman-Larhed and Artursson¹¹ on cocultures of Caco-2/HT29-H, using a mixture of different proportions of the cells at the time of seeding. Walter et al.¹² (Caco-2/HT29-MTX) and Meaney et al.⁹ (Caco-2/HT29GlucH) used only one ratio. Hilgendorf et al.¹³ selected three different Caco-2/HT29-MTX cell ratios, in single factor experiments. A coculture without serum in a single ratio of Caco-2/HT29-5M21 cells was developed by Nollevaux et al.¹⁴ Poquet et al.¹⁵ developed a coculture model with a single Caco-2/HT29-MTX cell ratio to analyse the transport of ferulic acid. The latest studies have used different Caco-2/HT29-MTX cell ratios to predict iron bioavailability.¹⁶ Chen et al., in recent work in 2010,¹⁸ studied various factors affecting the permeability of active substances in Caco-2/HT29-MTX coculture models. The authors showed that the length of culture and the culture medium have a considerable effect on TEER values and permeability coefficients. However, in all these studies, seeding with HT29-MTX cells was carried out simultaneously with seeding with Caco-2 cells.

There is therefore an obvious need to develop new in vitro intestinal barrier systems which are both more flexible and more realistic.

### OBJECT OF THE INVENTION

In the context of the invention, the Applicant has demonstrated that it is possible to modify the characteristics of enterocytes, particularly Caco-2 cells, by varying the time of seeding with goblet cells. It is thus possible, in an ingenious way, to control the important parameters of the cell culture obtained, particularly the transport and metabolism of the compounds, to obtain an in vitro system as close as possible to the real conditions of the intestinal barrier in vivo.

More specifically, the cell culture process according to the invention consists of:
seeding a suitable culture medium with enterocytes, and then
after a delay, seeding the medium containing the enterocytes that have begun to proliferate, with goblet cells.

Characteristically, and unlike the prior art, the seeding of the two cell types according to the invention is not, therefore, simultaneous.

Enterocytes, also called absorption cells or brush border cells, are characterised by their ability to exhibit properties of the small intestine in vitro, particularly in terms of transport for biopharmaceutical studies and analyses. The enterocytes are preferably Caco-2 cells. They may, for example, be the CBBel clone at passage 47, with the American Type Culture Collection (ATCC) reference number CRL-2102, which is itself derived from a clone of Caco-2, ATCC reference number HTB-37. In practice, the latter can be used at a passage between passage 55 and passage 65.

Goblet cells, also called mucus-producing cells, are characterised by their ability to produce mucus. As far as the goblet cells are concerned, to advantage they are HT29-MTX cells. The process for obtaining them has been described in publications by Lesuffleur et a/.^{3,4,5} They can, for example, be the cell line HT29-MTX¹⁰⁻⁶ M at passage 13, available from Dr Theela Lesuffleur of INSERM UMR S 938 in Paris, France. This line can be used preferably at a passage between passage 14 and passage 22. Alternatively, they can be E12 and D1 clones described in the publication by Behrens et al. (Behrens I, Stenberg P., Artursson P., Kissel T. (2001) Transport of lipophilic drug molecules in a new mucus-secreting cell culture model based on HT29-MTX cells. Pharmaceutical Research 18, 1138-1145) or HT29GlucH described in the publication by Meaney et al.⁹

In the context of this application, it has been shown that it is possible to modify the characteristics of the cell culture obtained by varying the timing of seeding the two cell types involved.

Unlike the prior art which advocated simultaneous seeding of the two cell types, the method according to the invention proposes seeding the two types of cell separately, in other words, at different times.

More specifically, the culture medium concerned is first seeded with enterocytes, preferably Caco-2 cells.

The culture medium is therefore advantageously suited for optimal growth of the enterocytes. In the case of Caco-2 cells, and as already described in the prior art, the medium is to advantage a complete nutrient medium such as DMEM (Dulbecco's Modified Eagle Medium). To even greater advantage, a growth factor is added to it, such as heat-inactivated foetal bovine serum (FBS), to advantage at a concentration of 15% (v/v).

The culture medium may also contain:
L-glutamine e.g. in the form of Gluta-Max™;
D-glucose, to advantage at a concentration of 4500 mg/L;
phenol red, to advantage at a concentration of 15 mg/L;
sodium pyruvate, to advantage at a concentration of 110 mg/L;
non-essential amino acids, to advantage at a concentration of 1% (v/v);
the antibiotics necessary to maintain the characteristics of the strains present, e.g. 100 μg/mL of streptomycin and 100 IU/mL of penicillin.

Cells are cultured to advantage at 37° C. in a moist atmosphere at 5% CO₂. The culture medium is to advantage changed twice a week, or each day at the end of culture.

The total cell culture time, for the invention and classically, is to advantage between 21 and 35 days after seeding with enterocytes, to greater advantage between 21 and 30 days.

The second type of cells, i.e. the goblet cells, is not therefore seeded simultaneously, but on the contrary at a later time relative to the first cell type, i.e. the enterocytes. In practice, the goblet cells are seeded after the enterocytes.

More precisely and advantageously, this should not be until after the enterocytes have begun their cycle of cell division. In other words, the enterocytes must have started to proliferate. To even greater advantage, in order to see a difference from the coseeding, the second cell type is seeded at least one day (24 hours) after the first cell type.

As has already been said, the goblet cells are seeded in the medium containing the enterocytes which have started to proliferate. Seeding is preferably performed when the enterocytes form a monolayer. Nevertheless, seeding the second cell type should not be too late, either, and should lead, in particular, to mucus production.

In practice, the goblet cells are to advantage seeded on the surface of the enterocytes. To even greater advantage, the goblet cells are seeded directly onto the monolayer of enterocytes.

The time needed for the monolayer of enterocytes to form depends in practice on the conditions of enterocyte culture, particularly on the initial enterocyte seeding density. To modify the properties of the coculture obtained further, it is possible to adjust both the enterocyte seeding density and the time of seeding the goblet cells. Other factors may influence the enterocyte growth rate, including the growth medium and the culture temperature.

Under the conditions of this application, it was observed that it was particularly suitable to seed goblet cells between 1 and 8 days after seeding the enterocytes, preferably between 2 and 6 days, or even between 2 and 3 days. The coculture obtained, which produces mucus, has characteristics that are thus intermediate between the two cell types, particularly in terms of the transport and metabolism of compounds, and therefore tends to reflect the real in vivo conditions.

Moreover, the two cell types are seeded in a controlled manner. For this application, the following culture conditions proved to be particularly suitable:
enterocyte seeding density between 5,000 and 30,000 cells for a culture area of 0.33 cm², i.e. 15,000 to 90,000 cells/cm², to advantage 15,000 cells/cm²;
goblet cells seeded to advantage at 10⁴ cells for a surface area of 0.33 cm² i.e. 3.10⁴ cells/cm².
   It should be noted that these data relate to the initial seeding conditions for each cell type.

Moreover, the cells, particularly the enterocytes, are classically seeded onto a permeable culture insert, for example onto a polycarbonate filter. The Transwell™ system is particularly suitable for implementing the method according to the invention.

In another embodiment, this invention also concerns the cell culture which is likely to be obtained using the process described above, particularly after 21 to 35 days, or even 21 to 30 days following seeding the enterocytes. Cocultures thus produced are new, since it has been shown in this application that the seeding sequence has an effect on various characteristics (such as efflux, paracellular transport, etc.) which thus distinguish them from cultures with only one cell type or even from a coculture obtained after simultaneous seeding.

To advantage, the cell culture thus obtained has at least one of the following characteristics:
contains enterocytes and goblet cells;
mucus is produced,
a TEER (Trans Epithelial Electrical Resistance) value between that of the enterocytes and that of the goblet cells, resulting in a capacity for paracellular transport between that of the enterocytes and that of the goblet cells;
P-gp (P-glycoprotein—multidrug resistance gene-1) activity between that of the enterocytes and that of the goblet cells, resulting in a capacity for transcellular transport of P-gp substrate molecules between the capacity of the enterocytes and that of the goblet cells;
cytochrome CYP3A4 activity between the enterocyte activity and that of the goblet cells.

To advantage, the cell culture is differentiated by its TEER value and/or P-gp activity and/or cytochrome CYP3A4 activity, to advantage by its P-gp activity. As shown in this application, this is particularly marked when the goblet cells are seeded after an enterocyte monolayer has formed, typically between 2 and 8 days after seeding.

To advantage, the TEER value and/or P-gp activity and/or cytochrome CYP3A4 activity obtained using the coculture according to the invention differs from the TEER value obtained for a coculture produced under the same conditions but with simultaneous seeding of the two cell types.

In conclusion, the in vitro model which has been developed in the context of this application has two major advantages. Firstly, it allows the functional capacity of monolayers to be modified, particularly expression of P-gp (P-glycoprotein—multidrug resistance gene-1) and paracellular transport, to make them as close as possible to in vivo conditions. Furthermore, the monolayer is covered by a layer of mucus as in the natural intestinal barrier.

Such a cell culture can thus be used as an in vitro model of the intestinal barrier.

This cell culture may in particular allow bioavailability to be assessed, including the transport and/or metabolism of a compound of interest, especially an active substance or nutrient.

As is apparent from this description, using the method according to the invention the following aspects of a coculture consisting of enterocytes and goblet cells can be modified:
Production of mucus;
Transepithelial electrical resistance (TEER) and therefore paracellular transport;
Expression and activity of P-gp (P-glycoprotein—multidrug resistance gene-1) and therefore the efflux and the transcellular transport of active molecules which are P-gp substrates;
Expression and activity of cytochrome CYP3A4 and consequently the cellular metabolism of active substances that are metabolised in the intestines.

### BRIEF DESCRIPTION OF FIGURES

The way in which the invention can be implemented and the advantages ensuing from it are best illustrated by the non-exhaustive examples below, provided for information purposes, supported by the attached figures, where:

FIG. 1 shows the TEER values against time for Caco-2, HT29-MTX and different cocultures. Each graph corresponds to one experiment.

FIG. 2 shows the Papp values for Lucifer yellow (LY) in different monolayers. Each graph corresponds to an experiment.

FIG. 3 shows the Papp values for rhodamine 123 (Rho123) in the basolateral-apical direction.

FIG. 4 shows the influence of verapamil, a P-gp inhibitor, on rhodamine 123 (Rho123) in the basolateral-apical direction.

An asterisk (*) indicates the values obtained for the cocultures, which are statistically different from values obtained with each strain.

FIG. 5 shows the paracellular passage of Lucifer yellow from the apical compartment to the basolateral compartment for 2 initial Caco-2 densities.

FIG. 6 shows the Papp values for rhodamine 123 without verapamil in the basolateral-apical direction for two initial Caco-2 densities.

FIG. 7 shows the Papp values for rhodamine 123 (Rho 123) without verapamil in the basolateral-apical direction, for different cultures.

### EXAMPLES OF EMBODIMENTS OF THE INVENTION

### I) Method

### 1) Caco-2/HT29-MTX Seeding in a Transwell® System.

Two types of cells were used for this work. The CBBel clone of Caco-2 cells was obtained from the American Type Culture Collection (ATCC) at passage 47 and was used in experiments at passages 55 to 65. The cell line HT29-MTX¹⁰⁻⁶ M was provided by Dr Thecla Lesuffleur of INSERM UMR S 938, Paris, France, at passage 13 and was used in experiments at passages 14 to 22.

The two cell types were grown on a routine basis in 25 or 75 cm³ culture flasks maintained at 37° C. in a humidified atmosphere of 5% CO₂ in a complete culture medium of DMEM (Dulbecco's Modified Eagle Medium) containing GlutaMAX™, D-glucose (4500 mg/L), sodium pyruvate (110 mg/L) and phenol red (15 mg/L). 15% foetal bovine serum (heat-inactivated FBS), 1% non-essential amino acids and 1% antibiotics (100 μg/mL streptomycin and 100 IU/mL penicillin) were added to the DMEM. The medium was changed twice a week. The cells were subcultured when confluence of about 70-80% was reached. A trypsin-EDTA mixture was used to detach the cells at a concentration of 0.125 and 0.25% for HT29-MTX and Caco-2, respectively.

The Transwell™ (TW) system, which was used for the coculture, consisted of a 24-well plate with polycarbonate membrane filtering inserts (diameter: 6.5 mm; area of membrane: 0.33 cm²; pore size: 0.4 μm; 10⁸ pores/cm²; membrane thickness: 10 μm). Before seeding, the Transwell™ inserts were equilibrated at 37° C., 5% CO₂ with culture medium (upper compartment: 100 μL, lower compartment: 600 μL) for at least 1 hour. The medium was then aspirated and the cells seeded.

The day of Caco-2 seeding was considered as day 0 (D0). Caco-2 cells were seeded at a density of 3.10⁵ cells/mL in the upper compartment (3.10⁴ cells/0.33 cm² or 9.10⁴ cells/cm²). The lower compartment was then filled with culture medium. For the coculture, HT29-MTX cells were seeded at various times between day 0 and day 18 (D0 to D18) after the Caco-2 seeding. For day 0 (D0), the Caco-2 and HT29-MTX were seeded simultaneously by taking 100 μL of a cell suspension with concentrations of 3.10⁵ cells/mL of Caco-2 cells and 1.10⁵ cells/mL of HT29-MTX cells. For other times (D4, D6, D8, D10, D12, D14 and D18), the HT29-MTX cells were seeded directly onto the Caco-2 monolayers at a concentration of 1.10⁵ cells/mL (100 μL/insert), after removing the culture medium from the upper compartment. The Caco-2/HT29-MTX ratio was therefore 75/25 for each coculture. Seeding was performed in quadruplicate. Controls were made with monolayers of Caco-2 and HT29-MTX only. The culture medium was changed every two days for the first two weeks of culture. From the third week of culture until the transport experiments, the growth medium was changed every day. Monolayers were used for transport experiments between days 21 and 30 after Caco-2 seeding.

### 2) Checking the Functional Capacity of the Monolayers

### 2-1) Measuring the TEER:

The transepithelial electrical resistance (TEER) was determined using the Millicell-ERS (Electrical Resistance System), which is a special voltohmmeter for measuring the resistance of monolayers of cultured cells. This device measures the health of cell monolayers qualitatively and cell confluence quantitatively. To take the measurement, ‘chopstick’ electrodes (Ag/AgCl) of different lengths were immersed in a prewarmed culture medium (incubator at 37° C., 5% CO₂) of the two compartments of the Transwell™ insert. The TEER was monitored every two days after the sixth day of culture and before and after each transport experiment. The final resistance of the monolayer of cells was calculated by entering the net TEER value in the following equation:

TEER [Ω*cm²]=(R_{Transwell}−R_{blank})*A

R_{blank} was determined before each measurement in a Transwell™ insert without cells, maintained in the same conditions as the inserts containing cells. The R_{Transwell} value is the total resistance of the cell monolayer and the polycarbonate membrane. A is the area of the filter (0.33 cm²).

### 2-2) Permeability Studies:

The apparent permeability Pₐₚₚ (10⁻⁶cm/s) describes the absorption permeability.¹⁷ Papp is calculated using the following equation:

Pₐₚₚ[10⁻⁶cm/s]=dQ/dt*1/(A*C₀)*10⁶

dQ/dt [μmol/sec] is the flow of active substance across the cell monolayer over time.
A is the area of the membrane in cm².
C₀ is the initial concentration of active substance in the donor compartment (μmol).

The transport buffer (TB) used for all permeability experiments was composed of 25 mM D-(+)-glucose and 10 mM HEPES (4-(2-hydroxyethyl)-1-piperazineethanesulphonic acid) in a Hank's balanced salt solution, adjusted to pH=7.4 with sodium hydroxide (1 M). The solution was then filtered with a 0.20 μm filter. The TEER of the monolayers was measured before and after each transport study to check the integrity of the monolayer.

For the transport studies, permeability markers including Lucifer yellow (LY), propranolol and rhodamine123 (Rho123) were used as follows:
a) Lucifer yellow was used as a marker of paracellular permeability. LY transport was tested in the apical to basolateral direction. 100 μL of test solution at 50 μg/mL was introduced into the apical chamber. The basolateral chamber was filled with 600 μL of TB. After incubating for 120 min at 37° C., 5% CO₂, all the apical and basolateral samples were collected for measurement. The LY paracellular marker was analysed quantitatively using fluorescence analysis. The apical samples were diluted 1/100 in TB, while the basolateral samples were not modified. A black 96-well plate was filled with 100 μL of each sample and measured using a fluorescence plate reader (Perkin Elmer) with an excitation wavelength of 405 nm and an emission wavelength of 535 nm. The corresponding LY standards for the range 0.0156-8 μg/mL (R²>0.99) were analysed simultaneously.
b) Rhodamine 123, a P-gp substrate, was incubated simultaneously with propranolol at a concentration of 5 μM. The transport studies were performed in both directions. Rho123 was quantified by fluorometric analysis. The samples and dilution series were put into a 96-well black plate (100 μL) which was analysed using a fluorescence plate reader (Perkin Elmer) with an excitation wavelength of 485 nm and a wavelength of 535 nm for emission. The samples from the donor compartment were diluted (1/10) in TB, while samples from the acceptor compartment were not diluted. Standards for the range from 0.008-1 μM (R²>0.99) of Rho123 were analysed to obtain a calibration curve. Studies were performed with Rho123 in the presence of verapamil, a P-gp inhibitor. Experiments were only performed in the basolateral-apical direction. In short, 5 μM of Rho123 and 100 μM of verapamil were added to the basolateral chamber. At the same time, the apical chamber was filled with 100 μM of verapamil. The transport study was performed under exactly the same conditions as the experiments with Rho123 alone.

### II) Results

### 1) TEER Values

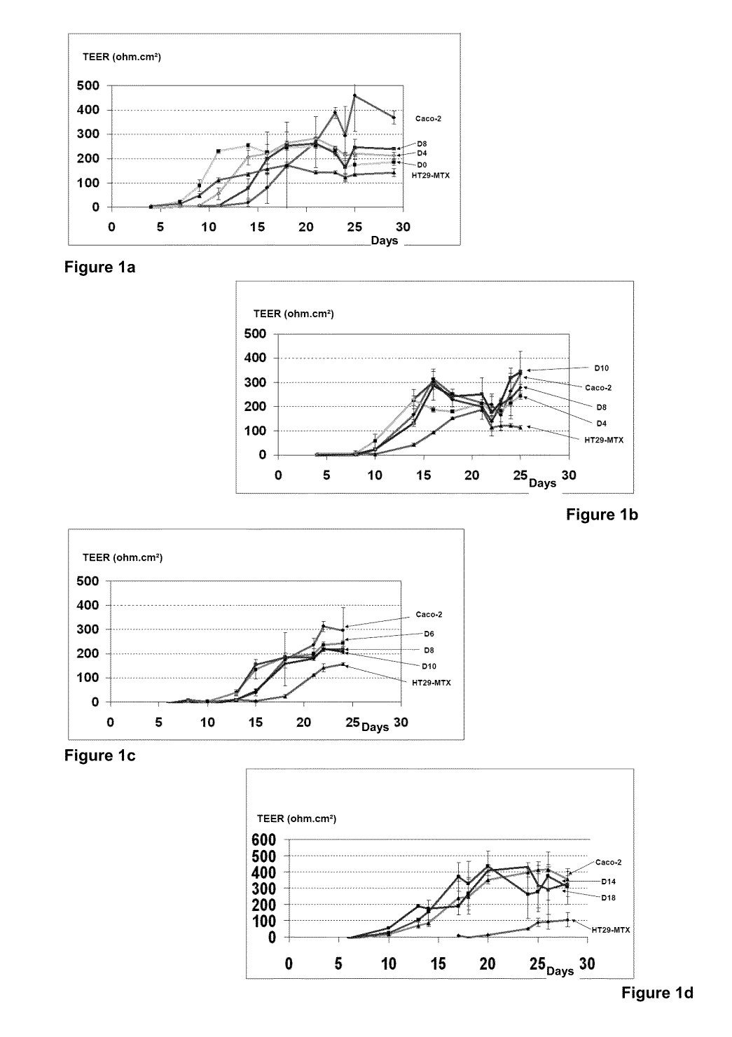

The results obtained in this series of experiments are presented in FIG. 1. FIG. 1 shows that the majority of Caco-2 monolayers have TEER values greater than 250 ohm.cm² 21 days after seeding, while lower TEER values are seen with HT29-MTX alone. The value of 250 ohm.cm² is a threshold value which proves the presence of tight junctions. The same level of TEER was also obtained with cocultures on days 14 (D14) and 18 (D18), suggesting that the density of HT29-MTX cells in cocultures is very low. In contrast, for earlier seeding times HT29-MTX (D6 and D8), TEER values are intermediate between those of the Caco-2 and HT29-MTX monolayers alone.

### 2) Paracellular Transport

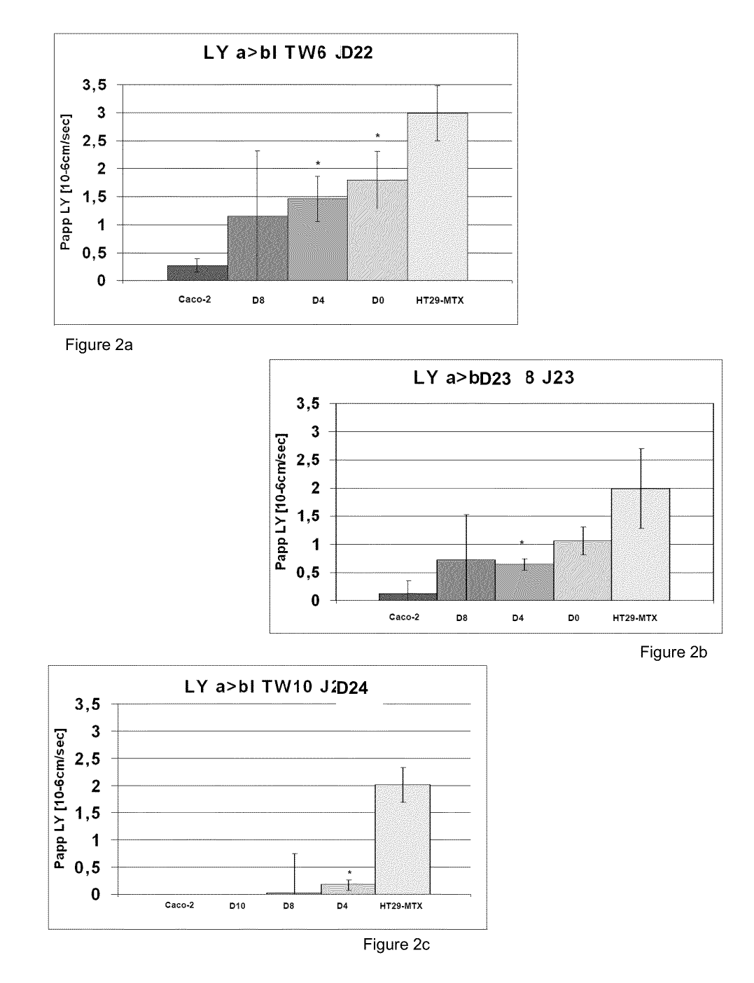

The data concerning these experiments are shown in FIG. 2.

In line with the literature, an LY Pₐₚₚ of less than 0.4 (10⁻⁶ cm/s) is considered as being acceptable for a monolayer of Caco-2. The paracellular permeability is explained by the presence of tight junctions between the cells. In contrast, the Pₐₚₚ for HT29-MTX is much higher with values around 2-3 (10⁻⁶ cm/s). Adding HT29-MTX to Caco-2 cells drastically increases the Pₐₚₚ. A direct relationship can be seen between the Pₐₚₚ values and the time of HT29-MTX seeding. HT29-MTX cells may therefore facilitate the paracellular transport of molecules, which can be adjusted depending on the day of HT29-MTX seeding. These results agree perfectly with the TEER values.

### 3) Rhodamine 123

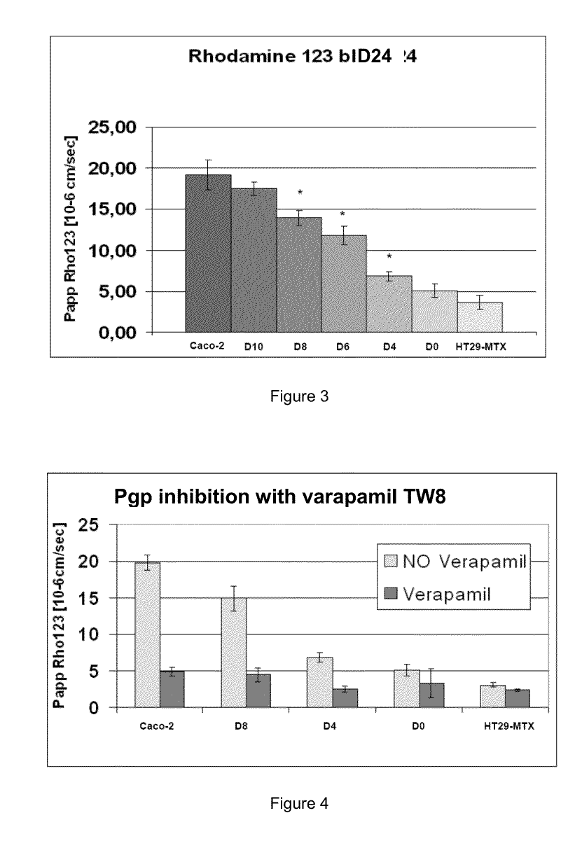

The results for these experiments are shown in FIG. 3.

In FIG. 3, the Rho123 Pₐₚₚ of a Caco-2 monolayer is much higher than that of HT29-MTX, with values of 19 and 3.10⁻⁶ cm/s, respectively. In contrast to HT29-MTX, P-gp is upregulated in Caco-2 cells, explaining the high efflux activity. As for the paracellular permeability study, the level of efflux transport depends on the day of HT29-MTX seeding. The earlier the HT29-MTX seeding, the more the Pₐₚₚ decreases.

The effect of verapamil, a P-gp inhibitor, is shown in FIG. 4.

In this figure, when verapamil is added to the monolayers, the Pₐₚₚ values for all the monolayers are similar, demonstrating the absence of efflux. Additionally, no significant difference was observed with HT29-MTX with or without verapamil, suggesting that expression of P-gp is absent in this strain. This explains why the presence of HT29-MTX in the coculture significantly reduces efflux due to a lower level of P-gp expression.

### 4) Influence of the Initial Caco-2 Density:

Additional experiments were performed to show the possibility of modifying the characteristics of the coculture obtained even more, depending on the initial Caco-2 density:
with an initial seeding of 5,000 cells/insert;
with an initial seeding of 10,000 cells/insert.

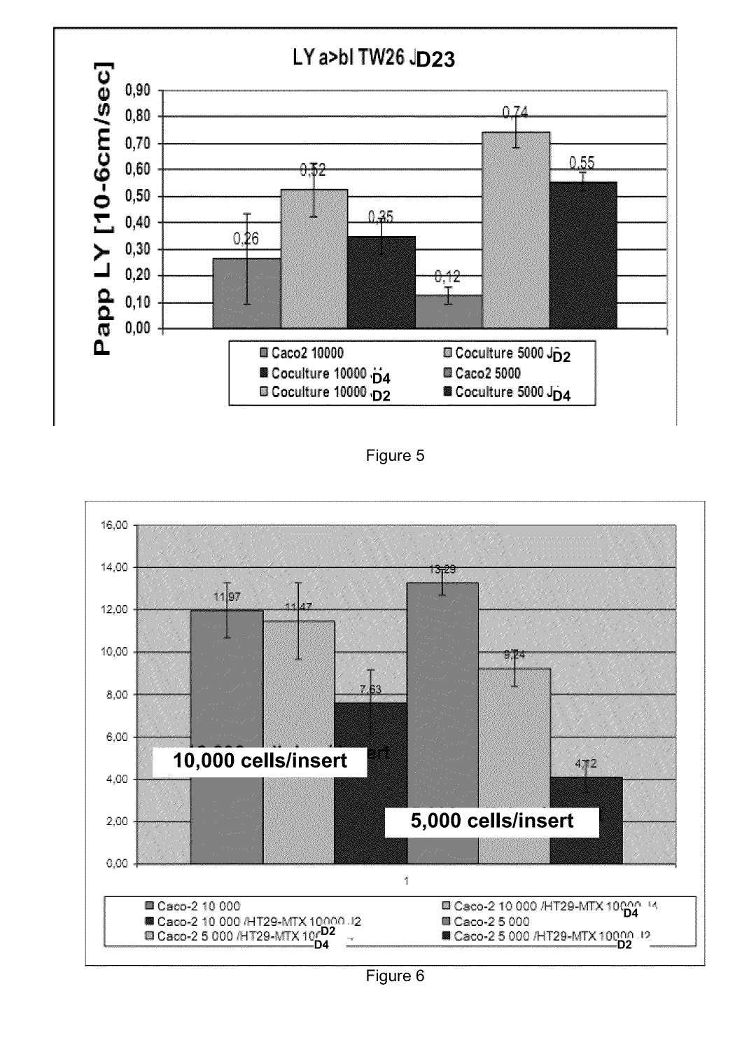

FIG. 5 shows the effect of the initial Caco-2 density on the time necessary before the second cell line is seeded on the paracellular passage of Lucifer yellow. By decreasing the initial density, it is possible to differentiate better the coculture according to the invention from the Caco-2 culture, particularly on day 2.

FIG. 6 shows the impact of the initial Caco-2 density on the time before seeding with HT29-MTX cells, in the event of transport of rhodamine 123 without verapamil. It can be seen that with a Caco-2 cell density of 10,000 cells per insert, the efflux activity of the coculture is different from that of Caco-2 cells alone on D2 but not on D4. On the other hand, with a Caco-2 cell density of 5,000 per insert, the cocultures performed at D2 and D4 are different from cultures of Caco-2 alone. The time necessary before HT29-MTX seeding can therefore be modified according to the initial Caco-2 density.

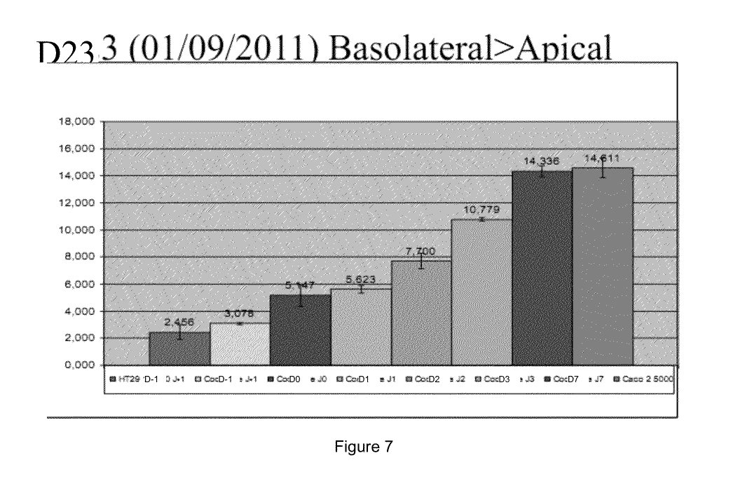

FIG. 7 clarifies the particular case where the initial Caco-2 cell density was 5,000 cells per insert: note that the coculture with separate seeding of the two cell lines differs from Caco-2, HT29-MTX and D0 when the time before seeding the HT29-MTX cells is between 2 and 3 days after Caco-2 seeding (5,000 cells per insert).

### REFERENCES

1—I. J. Hidalgo, J. Li (1996) Carrier-mediated transport and efflux mechanisms in Caco-2 cells. Adv. Drug Del. Rev., Vol. 22, Issues 1-2, 53-66
2—A. Quaroni, J. Hochman (1996) Development of intestinal cell culture models for drug transport and metabolism studies. Adv. Drug Del. Rev., Vol 22, 3-52
3—T. Lesuffleur, A. Barbat, E. Dussaulx, A. Zweibaum (1990) Growth Adaptation to Methotrexate of HT-29 Human Colon Carcinoma Cells Is Associated with Their Ability to Differentiate into Columnar Absorptive and Mucus-secreting Cells. Cancer Res., Vol. 50, 6334-6343
4—T. Lesuffleur et al. (1991) Dihydrofolate Reductase Gene Amplification-associated Shift of Differentiation in Methotrexate-adapted HT29 Cells. J. Cell. Biol., Vol. 115, 1409-1418
5—T. Lesuffleur et al. (1993) Differential expression of the human mucin genes MUC1 to MUC5 in relation to growth and differentiation of different mucus-secreting HT-29 cell subpopulations. Journal of Cell Sciences, Vol. 106, 771-783
6—J. Hunter, B. H. Hirst, N. L. Simmons (1993) Drug Absorption Limited by P-Glycoprotein Mediated Secretory Drug Transport in Human Intestinal Epithelial Caco-2 Cell Layers. Pharm. Res., Vol. 10, No. 5, 743-749
7—E. Le Ferree et al. (2001) In Vitro Models of the Intestinal Barrier. E. ATLA. Vol. 29, 649-668
8—R. A. Cone (2009) Barrier properties of mucus. Adv. Drug Del. Rev., Vol 61, Issue 2, 75-85
9—C. Meaney, C. O'Driscoll (1999) Mucus as a barrier to the permeability of hydrophilic and lipophilic compounds in the absence and presence of sodium taurocholate micellar systems using cell culture models. European Journal of Pharm. Sciences, Vol. 8, 167-175
10—A. Wikman-Larhed, P. Artursson, E. Bjork (1998) The Influence of Intestinal Mucus Components on the Diffusion of Drugs. Pharm. Res., Vol. 15, No. 1, 660-665
11—A. Wikman-Larhed, P. Artursson (1995) Co-cultures of human intestinal goblet (HT29-H) and absorptive (Caco-2) cells for studies of drug and peptide absorption. Eur. J. Pharm. Sci., Vol. 3, 171-183
12—E. Walter et al. (1996) HT29-MTX/Caco-2 Cocultures as an in Vitro Model for the Intestinal Epithelium: In Vitro-in vivo Correlation with Permeability Data from Rats and Humans. Journal of Pharmaceutical Sciences, Vol. 85, No. 10, 1070-1076
13—C. Hilgendorf et al. (2000) Caco-2 versus Caco-2/HT29-MTX co-cultured cell lines: Permeabilities via diffusion, inside- and outside-directed carrier-mediated transport. J. Pharm. Sci., Vol 89, Issue 1, 63-75
14—G. Nollevaux et al. (2006) Development of a serum-free co-culture of human intestinal epithelium cell-lines (Caco-2/HT29-5M21). BMC Cell Biology, 7:20, 1-11
15—L. Poquet, M. N. Clifford, G. Williamson (2008) Transport and Metabolism of Ferulic Acid through the Colonic Epithelium.Drug Metabolism and Disposition, Vol. 36, No. 1, 190-197
16—G. J. Mahler, M. L. Shulter, R. P. Glahn (2009) Characterization of Caco-2 and HT29-MTX cocultures in an in vitro digestion/cell culture model used to predict iron bioavailability. Journal of Nutritional Biochem., Vol 20, 494-502
17—P. Artursson, J. Karlsson (1991) Correlation between oral drug absorption in humans and apparent drug permeability coefficients in human intestinal epithelial (Caco-2) cells. Biochem Biophys Res Commun, Vol. 175, No. 3, 880-885
18—X. M Chen, I. Elisia, D. D. Kitts (2010) Defining conditions for the co-culture of Caco-2 and HT29-MTX cells using Taguchi design. Journal of Pharmacological and Toxicological Methods, Vol. 61, 334-342

1/ Cell culture process comprising:
seeding a culture medium with enterocytes and then
after a delay, seeding the medium containing the enterocytes that have begun to proliferate, with goblet cells.

2/ Process according to claim 1, wherein the enterocytes comprise Caco-2 cells.

3/ Process according to claim 1, wherein the goblet cells comprise HT29-MTX cells.

4/ Process according to claim 1, wherein the goblet cells are seeded at least 1 day after seeding the enterocytes.

5/ Process according to claim 1, wherein the goblet cells are seeded when the enterocytes form a monolayer.

6/ Process according to claim 1, wherein the cells are cultured for 21 to 35 days.

7/ Process according to claim 1, wherein the culture medium comprises DMEM.

8/ Process according to claim 1, wherein the enterocytes are seeded at a rate of 15.103 cells/cm2 and the goblet cells at a rate of 3.104 cells/cm2.

9/ Process according to claim 1, wherein the enterocytes are seeded on a permeable culture insert.

10/ Process according to claim 1, wherein the goblet cells are seeded on the surface of the enterocytes.

11/ (canceled)

12/ (canceled)

13/ (canceled)

14/ (canceled)
